(19) United States
(12) Patent Application Publication
(10) Pub. No.: US 2007/0095352 A1
(43) Pub. Date: May 3, 2007

(54) Endotracheal tube with markings

(76) Inventor: Jonathan Berall, Brooklyn, NY (US)

Correspondence Address: Berall, Jonathan, 173 Columbia Heights, Brooklyn, NY, 11201, US

(21) Appl. No.: 11/544,915

(22) Filed: Oct. 7, 2006

Related U.S. Application Data
(60) Provisional application No. 60/728,039, filed on Oct. 18, 2005.

(51) Int. Cl. A62B 9/06; A61M 16/00

(52) U.S. Cl. 128/207.15; 128/207.14

(57) ABSTRACT

Intubation is the placing of a plastic tube into the windpipe/trachea. The tube is placed into the trachea to facilitate giving oxygen to the patient. The plastic tube is called an endotracheal tube. Seeing the target, the windpipe opening, is critical to successful intubation. Likewise, seeing the tip-region of the tube to be inserted into the tracheal opening is important to intubation success. This patent will describe the marking of the distal/far-end region of an endotracheal tube so that the tip-cuff distal region will be more clearly seen against the brightly-lighted, light pink-orange tissues of the deep throat area to provide 1) clearer vision of the distal region of the endotracheal tube, 2) clearer vision of the endotracheal tube approaching and then entering into the windpipe opening, and 3) the ability to estimate the distance that the endotracheal tube has advanced into the trachea. This improved visualization of the distal region of the endotracheal tube will be particularly helpful with the new to market video laryngoscopes and other new, video-enhanced intubation instrument systems. The endotracheal tube will be held and manipulated and connected to oxygen exactly as it is at present.

### BACKGROUND OF THE INVENTION

The invention relates generally to endotracheal tubes, and more particularly to markings on the distal regions to include the tip-cuff and body region.

The invention is to mark/color, blacken, whiten the distal region of the endotracheal tube so that those forward elements of the endotracheal tube can be more easily and distinctly seen against the brightly-lighted, pink-orange background of the throat.

With the result that the endotracheal tube can be more accurately directed toward and into the windpipe opening, and its depth into the trachea estimated.

The marking will variably include metal markings so that x-rays of the tube placement will be more precise than is now possible.

The invention will be useful for the present intubation systems and also for the new-to-market, video-enhanced systems that help guide the endotracheal tube into the windpipe opening.

The result will be smoother, faster and more successful intubations.

The standard endotracheal tube is a hollow plastic tube that goes into the trachea to deliver oxygen. The adult size standard endotracheal tube has a diameter of about ½ inch, a tip of about 1 inch, a cuff of about 1½ inch, a body of about 12 inches, and a connector to oxygen of about 1 inch. There are different sizes to fit different patients; neonates to adults.

Presently endotracheal tubes are produced in clear or light plastic which must be seen against the brightly-lighted, pink-orange walls of the deep throat area.

No distinction is made between the distal region and the rest of the endotracheal tube that would allow the tip-cuff area to be more distinctly seen.

The clarity of the plastic of the endotracheal tube means that when the mouth is illuminated by the bright light on the laryngoscope blade, or the light sources on the other instruments which are used to intubate, the colors of the illuminated walls of the mouth will be seen through and behind the endotracheal tube, and the exact far region of the advancing tube is not distinctly seen.

### DISCUSSION OF THE PROCEDURE

Intubation:

placing a plastic breathing tube into a persons airway (windpipe/trachea) is done in emergency situations where the person is either not breathing or not breathing well enough and is about to have a catastrophic event such as a stroke or a heart attack. It is never done except in the most dramatic and potentially catastrophic situations.

Intubation is also performed in all general-anesthetic surgeries: >>10 million times in the U.S. each year, which represents ˜40% of the world total, after the anesthesiologist has removed the patients ability to breath spontaneously.

Seeing the target/the windpipe opening is the key to a successful intubation attempt. If the windpipe opening is not seen the intubation attempt will fail except rarely.

The target in an adult is about the size of a dime.

The circumference of the endotracheal tube is just a little bit smaller. The endotracheal tubes far end must enter the windpipe opening and pass through the vocal cords. Its far end is normally beveled to facilitate that entry.

A single attempt must be completed in 30 seconds, during which time the person is not receiving oxygen. If the attempt is not successful the person is given oxygen with bag/mask for 60 seconds and a second intubation attempt is made. If not successful another 60 seconds of bag/mask breathing is given, followed by a third and last attempt is made. If not successful other intubating instruments can be tried or the throat cut and a tube placed directly into the trachea. Already 3.5 minutes of inadequate oxygenation has passed. At 4 minutes brain cell damage is significant enough to be clinically apparent. Once the endotracheal tube has passed through the windpipe opening, the distance the endotracheal tube moves into the trachea is also important because the endotracheal tube cuff must sit past the vocal cords and not be so much advanced that the tube end passes the junction of the right and left lungs going into one lung; therefore not oxygenating the both lungs.

In performing an intubating procedure the professional intubator must first see the windpipe opening/the target.

The intubator then places the far end of the endotracheal tube into the windpipe opening and continues advancing the endotracheal tube down into the trachea.

If the windpipe opening is not seen by the intubator the intubation will fail except rarely, and tissues around the windpipe opening will be damaged making subsequent intubation attempts more difficult.

Big point: just as seeing the target is essential for successful intubation, seeing the end of the tube that is to enter the target is important for successful intubation: if you can see the target but you cannot distinctly see the end of the handheld tube you want to hit the target with, then you cannot know precisely in what direction to advance the tube.

With present endotracheal tubes the endotracheal tube tip-region is seen but it is not seen as well as it will be seen when the distal tip-region is marked so that it does not permit the illuminated mouth walls to be seen through and behind the endotracheal tube.

First intubation attempt success is the goal.

Any advantage that can be reasonably given to the intubator and to the patient should be given. More distinctly seeing the part of the endotracheal tube that is to go into the windpipe opening will be an advantage.

### RELEVANT PRIOR ART

Hippilito, R. B.

1) U.S. Pat. No. 6,889,693, May '05
2) U.S. Pat. No. 6,668,832, December '03
   these patents relate to endotracheal tube markings in the proximal regions of the endotracheal tube.

This patent relates to markings in the distal region of the endotracheal tube.

The Hippilito patents relate to estimating the position of the endotracheal tube once it has been inserted into the trachea using markings outside the mouth. The markings in this patent estimate the depth of the endotracheal tube in the trachea from inside the mouth. This patent relates to markings which facilitate the entry of the endotracheal tube into the trachea; and also to estimating the position of the endotracheal tube once it has been inserted into the trachea.
3) Heller, U.S. Pat. No. 4,567,882 February '86: illuminating the tip of the endotracheal tube. This patent approaches the problem of seeing where the distal tip of the endotracheal tube is. Its solution is technically complicated/relatively costly and of questionable value when used with video systems wherein the additional illumination will likely complicate rather then clarify the visual representation of the advancing endotracheal tube and the windpipe opening on the monitors.

### SUMMARY OF THE INVENTION

The primary objective of the present invention is to provide an endotracheal tube that will have its distal tip-region marked in such manner that 1) the distal region will be seen more clearly and more distinctly compared with the endotracheal tubes now in use; thus facilitating the entry of the endotracheal tube into the windpipe opening and, 2) will allow the intubator to estimate the depth of endotracheal tube once placed into the trachea.

A further object is to provide an endotracheal tube that is user friendly to the professional intubator: no changes in any other characteristics of the endotracheal tube except the proposed markings.

these and other objects and advantages will be apparent to those skilled in the art in light of the following disclosure, claims and accompanying drawings.

### BRIEF DESCRIPTION OF THE DRAWINGS

FIG. 1: Presents three representations of the standard endotracheal tube: with a tip region (1), a cuff region (2), a body region (3) and a connector to oxygen (4).

FIG. 1A represents the present standard endotracheal tube.

FIGS. 1B and 1C represent the standard endotracheal tube plus the modification of markings in the distal region to more distinctly see the distal/tip region, to facilitate the movement towards and placement into the windpipe opening, and also to allow the intubator to estimate how far into the trachea the endotracheal tube has gone once it has been placed into the windpipe opening.

### DESCRIPTION OF THE PREFERRED EMBODIMENT

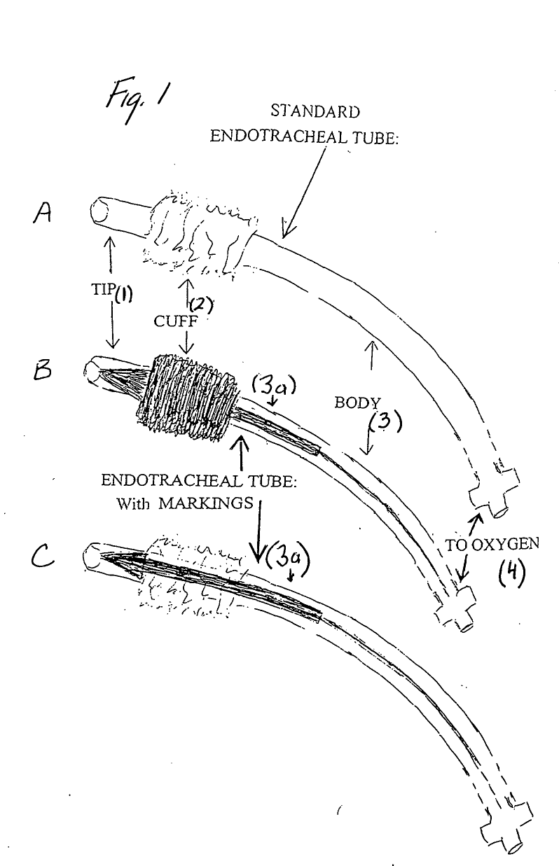

FIG. 1 presents the standard endotracheal tube (A) and the same endotracheal tube (A) plus markings in the distal region and the body (B, C).

In B and C the tip (1) and the cuff (2) and the body (3) of the endotracheal tube are variably marked; the commonality being that the distal region is better seen with the markings than without the markings.

The region (3a) proximal to the cuff (2) in B and C, in the distal region of the body (3) is distinctly separated from the remainder of the body (3) marking to facilitate the estimation of how far into the trachea the tip-cuff (1,2) has gone.

It will be understood by those skilled in the art that various deviations may be made in the shown embodiment without departing from the main theme of invention set forth in claims which follow:

I claim:

1) an endotracheal tube with the standard present design comprising:
a plastic tube of variable length and variable circumference; depending on the expected windpipe/trachea size of the patient, neonate-infant to adult.
having a proximal end to which is attached connections to oxygen and a distal end which enters the windpipe opening and advances into the trachea.
having a cuff around the tube in the distal region to secure the endotracheal tube in the trachea.
And additionally having markings in the distal region of the endotracheal tube that will allow the intubator see better the distal region of the endotracheal tube against the background of the illuminated mouth.

2) as in claim 1 with the tip and variably the cuff region marked to allow them to be more distinctly seen by the intubator than is now possible, as the endotracheal tube approaches and enters the windpipe opening.

3) As in claim 1 with the region proximal to the cuff so marked that the intubator can estimate the depth that the tip-cuff region has gone into the trachea.

4) the endotracheal tube as claimed in claim 1 wherein the markings are so designed that the distal end of the endotracheal tube suggests the appearance of the distal end of an arrow.

5) as in claim 3, these markings would have clearly demarcated segments within the region proximal to the cuff and would extend proximally from the cuff to a distance that would most importantly include the region where the endotracheal tube passes into the windpipe opening.

6) as in claim 1, the markings in the tip area can variably contain metal to improve x-ray evaluation of the endotracheal tube placement.
